(19) United States
(12) Patent Application Publication
(10) Pub. No.: US 2024/0086059 A1
(43) Pub. Date: Mar. 14, 2024

(54) Gaze and Verbal/Gesture Command User Interface

(71) Applicant: Luxsonic Technologies Inc., Saskatoon (CA)

(72) Inventors: John Corydon Huffman, Normandy Park, WA (US); Michal Wesolowski, Saskatoon (CA)

(21) Appl. No.: 18/462,154

(22) Filed: Sep. 6, 2023

Related U.S. Application Data
(60) Provisional application No. 63/375,306, filed on Sep. 12, 2022.

(51) Int. Cl. G06F 3/0487; G06F 3/01; G06F 3/04815; G06F 3/0482; G06F 3/04845; G06F 3/16; G16H 30/40

(52) U.S. Cl. CPC G06F 3/0487; G06F 3/013; G06F 3/017; G06F 3/04815; G06F 3/0482; G06F 3/04845; G06F 3/167; G16H 30/40

(57) ABSTRACT

User interface methods and systems are provided for interfacing with a plurality of objects in three-dimensional virtual environment providing for a medical imaging workspace. A method includes identifying one of a plurality of objects arranged in a three-dimensional virtual medical imaging workspace as a user-intended contextual selection based on detected gaze tracking information and establishing a command context based on the identified object. The command context comprises voice-activated commands, gesture-activated commands, or a combination thereof. The method further includes activating an object-specific action based on a detected voice or gesture command.

### RELATED APPLICATION

This application claims the benefit of U.S. Provisional Application No. 63/375,306, filed on Sep. 12, 2022. The entire teachings of the above application are incorporated herein by reference.

### BACKGROUND

Virtual reality (VR) systems, including extended reality (XR) and augmented reality (AR) systems, typically make use of head mounted displays (HMDs) to present a virtual, three-dimensional (3D) environment to a user. To interface with items present in the virtual 3D environment, users are provided with 3D mice. Such 3D mice include handheld controllers, which are moved about in free-space by the user, and desktop mice having joystick-like features to provide for additional degrees of freedom over 2D mice. With handheld controllers, motion of each controller is detected by the VR system to enable the user to navigate through the 3D environment, with additional inputs being provided by buttons included on the controller. In complex virtual environments, several objects may be present in the 3D space, which may thereby necessitate significant amounts of manipulation of the input device by the user.

Medical imaging reads and associated tasks are typically performed within imaging reading rooms at hospitals or other healthcare facilities. The read rooms at such facilities typically include workstations having one or more monitors to present both imaging and non-imaging data from multiple systems to the radiologist in a controlled environment. Recently, virtual medical imaging reading room environments have been developed, as described, for example, in International Pub. No. WO2022/047261, the entire teachings of which are incorporated herein by reference.

There exists a need for improved methods of interfacing with a virtual, 3D environment, particularly for virtual medical imaging reading room environments.

### SUMMARY

Methods and systems are provided that can enable more facile and less fatiguing interaction with objects in a 3D virtual environment, particularly in a medical imaging workspace environment.

A computer implemented method of interfacing with a plurality of objects in a three-dimensional virtual medical imaging workspace environment includes identifying a user intended contextual selected of a given object of the plurality of objects arranged in the three-dimensional virtual workspace environment based on detected gaze tracking information of the user. The method further includes determining a command context based on the identified selected object. The command context includes voice-activated commands, gesture-activated commands (as by a mouse), or a combination thereof. The method further includes activating an object-specific action based on a voice or gesture command identified from the determined command context. The voice or gesture commands may invoke a context-defined menu.

A system for interfacing with a plurality of objects in a three-dimensional virtual medical imaging workspace environment includes a processor, and a memory with computer code instructions stored thereon, the computer code instructions are configured to identify a user intended contextual selection of a given object of the plurality of objects arranged in the three-dimensional virtual workspace environment based on detected gaze tracking information of the user. The processor is further configured to establish a command context based on the identified selected object, the command context comprising voice-activated commands, gesture-activated commands, or a combination thereof, and activate an object-specific action based on a voice or gesture command identified from the determined command context.

For example, at least a subset of the plurality of objects can be or include imaging study panes. Activating an object-specific action can include invoking a hanging protocol for a selected imaging study, invoking an image manipulation tool, invoking a dictation annotation tool, or any combination thereof. The method can further include linking a location within a selected imaging study pane to data associated with a complementary study pane displayed in the virtual environment and displaying the linked data in the complementary study pane.

Alternatively, or in addition, at least a subsect of the plurality of objects can include imaging workflow panes. The imaging workflow panes can include, for example, a study navigation pane, a communication pane, a reference pane, a patient data pane, or any combination thereof.

Activating the object-specific action and include locking the identified object for primacy within the three-dimensional virtual environment based on a detected voice command. Activating the object-specific action and include invoking a dictation mode based on a detected voice command and terminating the dictation mode based on a detected gesture command.

### BRIEF DESCRIPTION OF THE DRAWINGS

The foregoing will be apparent from the following more particular description of example embodiments, as illustrated in the accompanying drawings in which like reference characters refer to the same parts throughout the different views. The drawings are not necessarily to scale, emphasis instead being placed upon illustrating embodiments.

FIG. 1 is a diagram of an example user interface system for a three-dimensional virtual environment providing for a medical imaging workspace.

FIG. 2 is a diagram illustrating a manipulated three-dimensional virtual environment with the example user interface system of FIG. 1.

FIG. 3 is a flowchart illustrating an example user interface workflow.

### DETAILED DESCRIPTION

Medical imaging workflows have recently been adapted and integrated into VR/AR environments to provide for more advanced visualization over conventional, two-dimensional (2D), monitor-based user interfaces and to facilitate collaboration among remotely-located physicians. Such VR medical imaging environments can also provide for improved workflows and enable improved accuracy with respect to data evaluation and diagnoses.

When moving from a traditional 2D, monitor-based user interface (UX) environment to a 3D VR/AR environment, there are a number of fundamental challenges that can arise. In a 2D environment, interaction with windows and objects on the screen can be easily managed with a traditional mouse pointer and associated mouse actions. The intrinsically planar (i.e., 2D) environment allows the traditional computer mouse to move between monitors and windows (panes) unambiguously.

When moving to a true 3D environment (e.g., as can be presented when a user is immersed in a VR/AR environment), objects are not constrained to exist in a perceptually flat landscape. In addition, there is not a physical constraint of a “monitor” present. The viewing space can be effectively infinite. The introduction of depth can provide for objects to be perceived as continuously “farther away” or “nearer” to the observer, as well as above, below, to the left, and/or to the right of where a traditional monitor boundary would be. Adjacency is not a fixed characteristic in 3D.

Traditional mouse interactions make an assumption of a planar field of view and alignment or overlap of objects. The extensions of this paradigm to 3D using 3D mice, or other hardware interfaces, introduces a number of problems. Such problems can become particularly cumbersome over long term use of a 3D mouse. For example, 3D mice, such as handheld VR controllers, can require moving the hands in free space to simulate spatial separation of objects, as well as to manipulate angles and positions. This can rapidly become fatiguing, and this paradigm is exacerbated by a need to define some hierarchical rules for interacting with objects “above”, “in front of”, “behind” or “occluded by” other objects. These rules are far from standardized, and the 3D mice available on the market today have widely differing implementations from both the hardware, and software (driver) perspective.

Another difficulty in using 3D mice hardware in these environments arises with respect to invoking contextual functionality while potentially observing non-local objects. For example, using traditional mouse paradigms, one can invoke a function through a mouse click—either directly or through a menu. The context of the mouse functionality is frequently contingent on the location of the mouse in the viewing environment.

In a 3D VR space, with objects at virtual distances and angles, and possibly surrounding an observer, the actions needed to “move” the mouse pointer to establish a context can be complex and impractical. With the varying depths of vision and associated depth of objects in the space under observation, identifying how a traditional mouse pointer should work is complicated, and the need to maintain a context while observing a result elsewhere becomes far more complicated when manipulating a 3D mouse in space versus a 2D mouse that can be “parked” at a location on the monitor and released.

A description of example embodiments follows.

The provided methods and systems can include and make use of multiple technologies within the AR/VR environment to overcome the above-noted difficulties. Virtual radiology reading room environments are described in WO2022/047261, the entire teachings of which are incorporated herein by reference. The methods and systems provided can be applied to or integrated with such virtual radiology reading room environments. The examples described herein are described with respect to a VR environment; however, it should be understood that such methods and systems can be applied to any variation on a virtual 3D space, including AR and XR environments.

I. Use of Gaze to Establish Functional Context

An objective of the provided methods and systems is to minimize or eliminate a traditional mouse paradigm for navigating amongst objects in the VR environment. As the physical constraints of one or more 2D monitors are not present, a more flexible mechanism for establishing functional context and/or selecting objects can be implemented. Gaze tracking (i.e., direct ray tracing of the focus of eye fixation) can be integrated into a VR HMD. For example, the VR HMD can include an integrated sensor, such as a camera to detect the position, movement, direction or a combination thereof, of the eyes of a user wearing the VR HMD. By using a gaze-tracking paradigm to establish a functional context in place of a traditional physical 2D or 3D mouse in the virtual medical imaging environment, the time and effort to change a focused context can become effectively zero, from a user's perspective. For example, there is no need to “drag” a mouse pointer across or through spatial boundaries to focus on a separate object (e.g., a separate window, tool, etc.).

In the VR display environment, there can be a number of objects such as panes containing images, renderings, or other data. These panes may be free positioned, or in a constrained configuration with other objects, for example, a window with four different panes containing data. As the VR space is three dimensional, these objects have an x-y position, and a z position, so their [virtual] relative distance from the viewer can be computed. The semantics of these objects is maintained by the hosted application managing the VR environment. That is, the application can discriminate objects based on contextually relevant information about the contents—what is the modality of the medical image, what is the body part being displayed, is this from the current or the prior study, etc.

The gaze tracking component of the VR headset results in a vector updated and delivered in real time from the VR system to the hosted application environment that describes the direction of the user's gaze. The hosting application, or the native VR system in some cases, can then compute the intersected objects.

The hosted application can then filter the continuous gaze vector information to compensate for unintended wandering of the user's gaze that may make the specific vector path ambiguous. The hosted application can then use the semantics of the intersected object to determine the relevant intersected object that will determine the functional context to be referenced. The user, through a voice command, gesture, or UX command, can then establish a persistent context for subsequent actions. For example, the user may select a specific pane and freeze the context to that pane; the user can then peruse other objects in the VR space without affecting the selected functional context. Some examples enable the user to select a pane with a specific view, establish a persistent context, then browse the available objects for another relevant view and command the system to “LINK” the views for comparison. Another example can include selection and automatic display with the use of voice commands, where when a specific pane is selected, a command of “LINK PRIOR VIEW” can automatically display the related view from a prior study adjacent to the current view for comparison. The persistent context can be cancelled at any time with a gesture, voice command, or UX command.

Using gaze as a context select mechanism can support an effectively immediate move of a mouse cursor to a different location. Optionally, a voice command can be provided to confirm selection of the focused context. The use of gaze tracking to establish a context selection can avoid the time and effort involved in dragging a mouse cursor across the environment, which can thereby also allow a user to maintain focus and attention on a relevant object.

For example, within a medical imaging workflow, a user can be in a study navigation worklist and, with a gaze input, select a study for review. This action can then invoke a hanging protocol that can provide an appropriate layout of the selected study's images and/or series in a diagnostic configuration. In a further example, the user can then focus their gaze on a particular image of the study and invoke a mouse transport through a voice command to avoid the need to drag the mouse through other windows and object views. The mouse transport can thereby provide for the further manipulation of the image or selection of tools.

Gaze tracking methods for detecting an object of interest in a three-dimensional space are generally known in the art. They involve tracking the motion of the eye and ray tracing until an object of interest is encountered. It should be understood that, although there are different methods used by different systems for eye tracking, they typically result in a continuously updated vector describing the path of the gaze; and as such, the embodiments described herein function with these different methods.

II. Use of Voice Commands in Functional Context

Another objective of the provided methods and systems is to mitigate the traditional need for mouse gestures (e.g., clicking, dragging, scrolling, etc.) to invoke functionality, either directly or through menus. Extensible lists of voice commands in the different functional contexts of the VR environment can be provided to omit or reduce interactions that would otherwise occur via mouse gestures. Furthermore, a complexity of free speech interpretation can be minimized by parsing for pre-identified commands. For example, where a traditional mouse-click to bring up an option menu may have been used, a voice command “OPTIONS” can invoke the same menu, with the functional context determined by the gaze-identified window or other object. The menu can then appear with further delineated voice commands as menu items that can be further invoked.

This mechanism can alleviate or eliminate the discrimination problem between dictation and command semantics. Many systems have tried to parse free speech and interactively discriminate what is intended as a command, versus what is intended as dictation. Using the aforementioned method, a voice command of “DICTATE ANNOTATION”, or similar command, within the functional context of a suitable gaze-selected object, can then invoke the free dictation version of the voice recognition system. The voice recognition in dictation mode can then return a string (e.g., word, sentence, paragraph, report, etc.), and can be terminated by a keyword, mouse-click, or gesture. Such dictation command can be available within only certain functional contexts, as established by gaze-tracking, and can be combined with gesture tracking, as further described, to mitigate problems relating to traditional dictation methods within medical imaging workflows.

Voice recognition methods and systems are generally known in the art. Examples of suitable voice recognition systems include products from Nuance and MultiModal, which are well understood in the industry.

III. Use of Free Space Gestures in Functional Context

To further mitigate the traditional need for mouse gestures to invoke functionality, gesture recognition can be included in the command structure of example methods and systems. It is possible to use a physical mouse to invoke functionality, but a system supporting gesture-invoked events can provide for far more flexibility with less fatigue for a user and can complement voice command within gaze-identified objects. A number of systems on the market today support gesture input in multiple ways—for example, through gloves with embedded sensors, external cameras, accelerometers, etc. An example of a suitable gesture recognition system that does not use gloves or other hardware to track hand movements and gestures is that found in the Microsoft Xbox game console.

Similar to the use of pre-defined voice commands described above, a set of gestures can be defined that can be interpreted differently based on the functional context established using gaze-selection and, optionally, voice command. For example, in the dictation example described in the prior section, a sweep gesture of a hand can be interpreted as the terminator for speech to be returned as voice-recognized string.

Particularly in a medical imaging workflow environment, where dictations are a part of a standard workflow and where a physician may be navigating among several images of a study during a read while simultaneously dictating a report, the combination of gaze-tracking to establish a functional context, voice recognition to confirm selection and invoke dictation, and gesture recognition to confirm or terminate processes can provide for a streamlined workflow with much less aggravation and fatigue than can be required by 2D or 3D mouse selections.

IV. Use of Gesture Interaction with Virtual Objects

The provided systems and methods can further include tracking interaction of hand gestures with virtual objects in the VR environment. For example, “grabbing” an object, “pushing” a button, “turning” a knob, “magnifying” an image, etc. are object manipulations that can be invoked by gesture recognition. While gesture recognition providing for the interaction of virtual objects is common in VR systems, it can be cumbersome and inefficient to employ in an environment supporting complex functionality for object presentation, manipulation and editing. In an orchestrated context, as described above, gestures can provide for an augmented component of an overall UX environment. For example, gesture interactions can be invoked in a limited manner in certain contexts.

V. Orchestrated UX Functionality

When the four aforementioned techniques are orchestrated within the VR environment, complex interactive functionality can be supported with limited or no additional mouse requirements. A 2D or 3D mouse can optionally be used and supported in such an environment for specialized functionality that maps to the provided functionality, but the orchestration of these techniques together can provide for the user/observer to interact with and manipulate objects in a natural, intuitive way, with near zero incurred UX overhead caused by additional device manipulation.

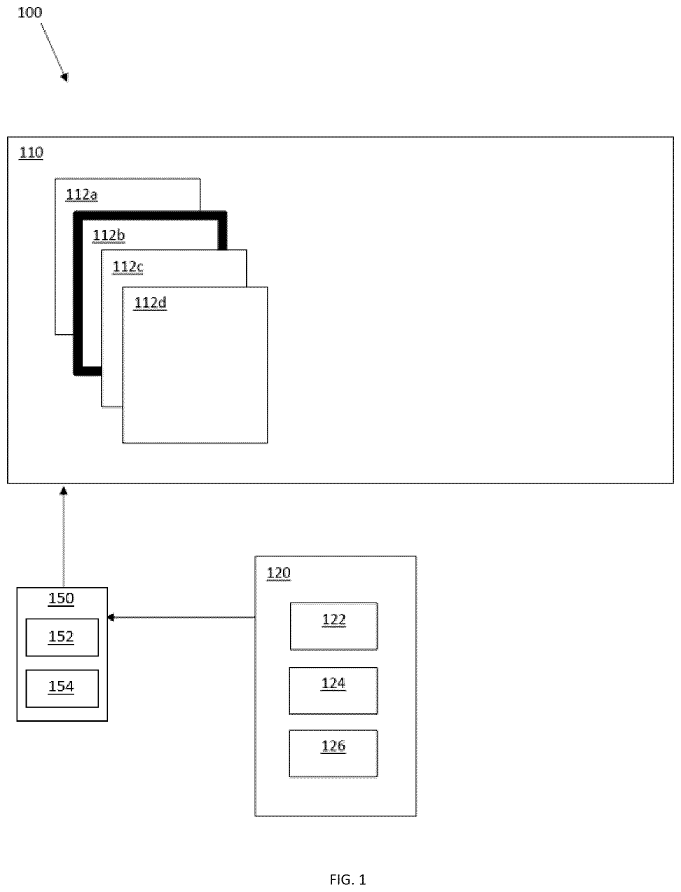

Example systems and methods are described to illustrate orchestration of gaze tracking, voice activation, and gesture activation in a medical imaging workflow environment. As illustrated in the system 100 of FIG. 1, a plurality of objects 112a, 112b, 112c, 112d are arranged in a 3D virtual environment 110 providing for a medical imaging workspace. A controller 150 is configured to receive user input 120, including gaze-tracking information 122, voice recognition information 124, and gesture recognition information 126. Controller 150 can include a processor 152, and a memory 154 with computer code instructions stored thereon. The controller is further configured to manipulate the 3D virtual environment 110 based on such user input 120.

For example, one of the plurality of objects (112b) can be identified as a user-intended contextual selection based on detected gaze tracking information 122. The controller can then be configured to establish a command context based on the identified object, the command context including object-specific, voice-activated and/or gesture-activated commands. An object-specific action based on the detected voice recognition information 124 and/or gesture recognition information 126 can then be invoked.

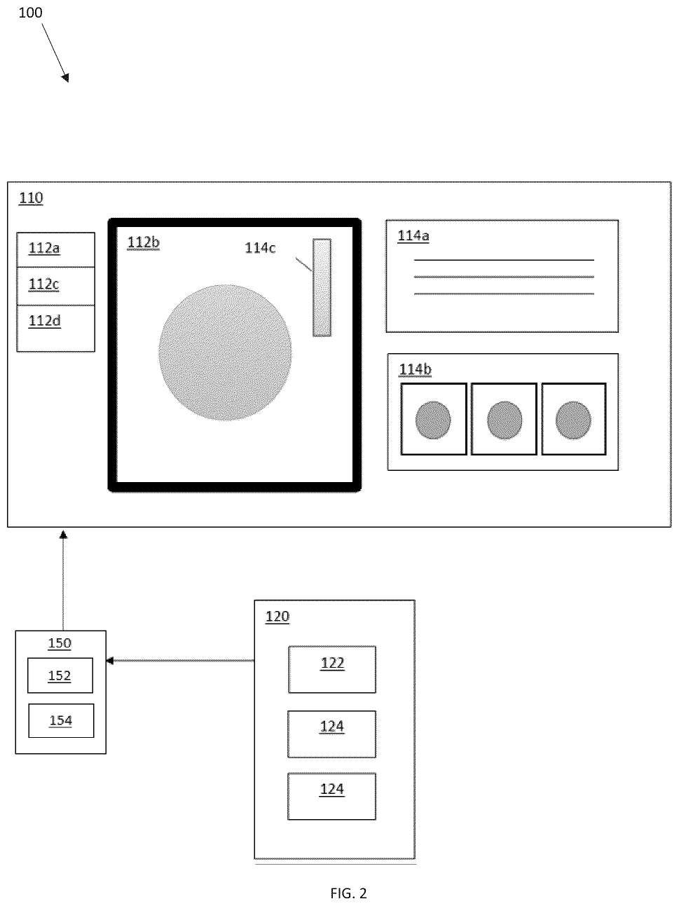

For example, the object 112b can be an imaging study pane. As further illustrated in FIG. 2, a voice command detected during gaze-selection of object 112b can prompt the system to lock the object 112b for primacy within the three-dimensional virtual environment (e.g., cause object 112b to “stick” within the user's field of view) and/or prompt a hanging protocol to be invoked for the selected study represented by object 112b. Alternatively, or in addition, other object-specific actions can be invoked by voice and/or gesture command, such as invocation of various tools 114a, 114b, 114c that may operate on the selected study, generate additional data for the study, and/or display information complementary to the study. Selection among the various tools can be invoked by further gaze-selection and/or voice/gesture commands. For example, an image manipulation tool 114c, such as a window contrast tool, may be activated by voice command, and manipulation of the tool 114c can be implemented by gesture commands to adjust contrast levels. In a further example, a dictation annotation tool 114a can be invoked by voice command, with gesture commands invoked to indicate a start and/or stop to a dictation mode.

In yet a further example, a location within the selected imaging study 112b (e.g., a particular anatomical location indicated by the displayed image, from within a series of images) can be selected and a complementary study pane 114b can be displayed in the virtual environment. The complementary study pane can display linked information to the selected study or to the displayed anatomical location within the selected study, for example, imaging data obtained from a same subject at earlier or subsequent timepoints to the selected study 112b, imaging data of a different modality for comparison, or reference images for comparison. The toggling of information in the complementary study pane can be invoked by further gaze-selection, voice and/or gesture commands.

The plurality of objects 112a-d can alternatively be imaging workflow panes, such as a study navigation pane, a communication pane, a reference pane, a patient data pane, or any combination thereof. Examples of applications and tools for display in a virtual reading room environment are further described in WO2022/047261, and any such items can be included or operated upon by the methods and systems described herein. Navigation among such objects using gaze-tracking and optional selection by confirmatory voice and/or gesture command can enable a physician to navigate through various workflow options.

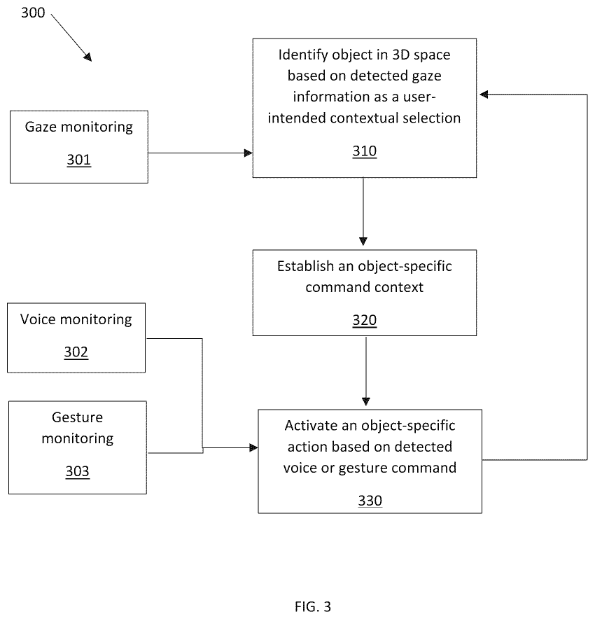

An example user interface method 300 is shown in FIG. 3. As illustrated, gaze monitoring 301, voice monitoring 302, and gesture monitoring 303 can be performed throughout user interaction with a 3D environment. An object in the 3D space can be identified (310) as a user-intended contextual selection based on detected gaze information. Based on the contextual selection, an object-specific command context can be established (320), and an object-specific action based on a voice and/or gesture command can be activated (330). Establishing an object-specific command context can include determining a command context based on an identified selected object, where the determined command context comprises voice-activated commands, gesture-activated commands, or a combination thereof. The process can optionally repeat as a user continues to work within a selected context or establishes a new contextual selection. The user interface method can further include displaying to the user the three-dimensional virtual medical imaging workspace, including the objects in the 3D space. Further, the user interface method can include identifying a command from the determined command context based on the detected user voice or gesture data.

The objects can be objects within a 3D workspace providing for a virtual medical imaging reading room or components thereof. Examples of objects existing within such a 3D workspace are further described in WO2022/047261, the entire contents of which are incorporated herein by reference. Each object can provide for one or more contextual selections by a user, such as, for example, selection of a particular imaging study, an imaging workflow pane, a study navigation pane, a communication pane, a reference pane, and a patient data pane. The objects can be virtual monitors derived from other systems, such as, for example, a Picture Archiving and Communication System (PACS), a Radiological Information System (RIS), and an Electronic Medical Record (EMR) system, or can be items presented within a virtual monitor.

Object-specific command contexts can be based on commands typically available by mouse-selection within a selected context. The following are examples of user interface workflows, with sample object-specific commands, that can be executed by a combination of gaze-tracking, voice, and gesture commands in a virtual medical imaging reading room context.

1. Establish Context Via Gaze Tracking and Invoke a Menu

Upon context selection via gaze-tracking, cascaded menu items can be invoked through voice commands. For example, where a user would typically execute a right-button mouse click to prompt a menu to cascade open, a voice command (e.g., “Options”) spoken within a selected context can prompt a menu to appear. The user can then invoke a desired option among the menu items via a further voice command that specifies the desired option or a mouse selection where the mouse has been moved to the opened menu to provide for ease of navigation. Cascaded menus can be invoked as per typical menu item functionality. Returning to a previous menu can be performed with a further voice command (e.g., “Back”), hand/arm gesture, or mouse gesture.

Where a menu item, or other invoked functionality, requires a free text field, the system can invoke a dictation mode for voice recognition. A termination gesture, such as a swipe of a hand, can conclude a dictation mode to alleviate issues that can arise with respect to parsing voice commands from voice dictation.

2. Establish Context Via Gaze Tracking and Invoke a Function Directly with Voice

For example, a voice command, such as “Window Width Level” can invoke a window adjustment (WW/WL) tool within an image viewing pane. Context-specific hand gesture commands can then be available to the user—for example, left-right movements can provide for window width adjustments, and up-down movements can provide for window level adjustments. A further voice or gesture command can terminate use of the tool.

3. Establish Imaging Context Via Gaze Tracking and Link Anatomical Locations with Voice

Where studies from multiple imaging modalities are available, such as, for example, a CT study and an MR study, a user can invoke a linking action via voice (e.g., “Link All”). For example, a user may select both a CT and an MR study for a given patient via a combination of gaze selection and voice command to cause both of the selected studies to “stick” within the user's field of view. A voice command such as “Link” can provide for localizing a same anatomical location within both studies. In a further example, a voice command, such “Lock Context,” can maintain the context, independent of further gaze context changes, until terminated with a further gesture or voice command. The locking function can enable a user to localize linked images across multiple windows or panes without generating confusion as to context selections.

Furthermore, imaging study interaction commands can be invoked. For example, a pre-set WW/WL value can be invoked with the established CT and MR context through use of a menu selection, as described above, or directly with a voice command, such as “bone window,” “lung window,” etc.

4. Organize Objects

A user can interact with objects/panes within the 3D environment without changing a functional linking of objects. In continuance of example item 3, above, a user may use a combination of voice and gesture commands to reorganize objects (e.g., an MR series, a scout image, etc.) during viewing and analysis without unlinking the imaging studies.

5. Functional Context by “Regions” of the Workspace

Various workspace regions can be selected and manipulated through gaze and voice/gesture command. For example, gaze can be utilized to establish a Communication panel as a contextual selection, with further functions, such as texting, invoked through menu selection and voice commands. In a further example, collaboration can be directly supported with local use of a voice command, such as “collaborate.”

In another example, gaze can be utilized to establish a Patient/Study Search panel as a contextual selection. Gaze can be further utilized to select particular fields, and entries dictated. For example, a user may establish a patient name field as a contextual selection and dictate a name to populate the field.

In yet another example, gaze can be utilized to contextually select a Research Links panel to search a topic of interest. For example, upon establishment of the Research Links panel as a contextual selection, a user may then provide voice commands for searching (e.g., “Look up examples of X,” “Look up definition of Y,” etc.).

In another example, navigation of a Patient Timeline can occur via gaze tracking. For example, additional patient studies can be selected for presentation via gaze selection. Alternatively, or in addition, voice commands can be used. For example, a voice command such as “Load prior” can invoke the presentation of an earlier-obtained study for the patient whose images are being viewed.

The orchestration of gaze-tracking selection with context-specific voice and/or gesture commands can enable a physician or other user to more easily and straightforwardly navigate a 3D virtual read room environment than by physical manipulation of a 3D mouse. Given the complexity of an imaging reading room environment, where multiple information sources, often from multiple, discrete manufacturer systems, are simultaneously presented to a user, use of a 3D mouse to concurrently navigate among the several sources is impractical and can be quickly fatiguing.

A user interface system that establishes a functional context based on gaze selection and enables object-specific actions to be invoked by voice and/or gesture commands can provide for much faster, less cumbersome, and less fatiguing interaction with objects in a 3D space, particularly for a medical imaging reading room environment.

A user interface in which gaze-tracking information establishes a functional context can include, for example, selection of a window or pane context (e.g., CT series, XR image, navigation pane, etc.), selection of open area context (e.g., generic system level functionality), and selection of an ancillary pane context (e.g., communication pane, reference data pane (Radiopedia, Wikipedia, etc.), report pane, patient history timeline, etc.)

A user interface in which predetermined voice commands invoke actions can include, for example, different endpoints for command audio streams and dictation audio streams. Optionally, an endpoint can be selectively invoked with a confirmatory gesture (e.g., a button click, hand gesture, etc.). Such user interfaces can be particularly advantageous in a medical imaging read room context where dictation is a standard component of imaging review

A user interface can require a combination of a voice command with a functional context to return an event with a requisite payload to invoke a specific action. The event can be intercepted by the administrative context of an application. An example includes providing an annotation to an imaging study. For example, a shape (circle, ellipse, etc.), a fixed label (pre-stored options for a context, e.g., tumor or mass labeling), or “free dictation” requiring an orchestration of the command and dictation endpoints can be invoked upon voice command within an imaging study context.

Another example includes linking series. By establishing the functional context by gaze at a particular series, linking can be invoked with all related series. This action can further align all localizers and series with the position of the selected series.

Yet another example includes miscellaneous actions, such as verbal commands (e.g., “pop-up”) to create a larger, un-occluded view of a selected image pane contents for analysis, (e.g., “close”) to close a pop-up window, close a study, load a next study, etc.

Another example includes report generation support. For example, selection of one or more relevant templates for dictation or auto-fill can be invoked.

Yet another example includes window positioning. For example, a verbal command such as “bring to front” within the context of overlapped or occluded windows can invoke a particular window, as identified by gaze selection, to be brought to a front of a user's field of view within the 3D space.

Patient-specific or study-specific actions can also be invoked, such as opening or closing prior studies. For example, context can be established with gaze on a patient timeline, and verbal commands, such as “open prior (optional descriptor)”, “close prior (optional descriptor”), etc., can invoke a specific action.

Such user interfaces can further include recognition of predefined manual gestures to perform context-specific actions, when appropriate. For example, scrolling (swipe left/right for next/previous), zooming (hand in/out), increase window size (use framing with fingers to indicate increase or decrease), etc.

The teachings of all patents, published applications and references cited herein are incorporated by reference in their entirety.

While example embodiments have been particularly shown and described, it will be understood by those skilled in the art that various changes in form and details may be made therein without departing from the scope of the embodiments encompassed by the appended claims.

What is claimed is:

1. A computer-implemented method of interfacing with a plurality of objects in a three-dimensional virtual medical imaging workspace environment, the method comprising, by a processor:
identifying a user intended contextual selection of a given object of the plurality of objects arranged in the three-dimensional virtual medical imaging workspace environment, based on detected gaze tracking information of the user in the three-dimensional virtual medical imaging workspace environment;
determining a command context based on the identified selected object, the determined command context comprising voice-activated commands, gesture-activated commands, or a combination thereof; and
activating an object-specific action based on a command identified from the determined command context.

2. The method of claim 1, wherein at least a subset of the plurality of objects comprises imaging study panes.

3. The method of claim 2, wherein activating an object-specific action comprises invoking a hanging protocol for a selected imaging study.

4. The method of claim 2, wherein activating an object-specific action comprises invoking an image manipulation tool.

5. The method of claim 2, wherein activating an object-specific action comprises invoking a dictation annotation tool.

6. The method of claim 2, further comprising:
linking a location within a selected imaging study pane to data associated with a complementary study pane displayed in the virtual environment; and
displaying the linked data in the complementary study pane.

7. The method of claim 1, wherein at least a subsect of the plurality of objects comprises imaging workflow panes.

8. The method of claim 7, wherein the imaging workflow panes comprise a study navigation pane, a communication pane, a reference pane, a patient data pane, or any combination thereof.

9. The method of claim 1, wherein activating the object-specific action comprises locking the identified object for primacy within the three-dimensional virtual environment based on a detected voice command.

10. The method of claim 1, wherein activating the object-specific action comprises invoking a dictation mode based on a detected voice command and terminating the dictation mode based on a detected gesture command.

11. The method of claim 1, further comprising, displaying to a user, the three-dimensional virtual medical imaging workspace including the plurality of objects.

12. The method of claim 1, further comprising, identifying a command from the determined command context based on the detected user voice or gesture data.

13. A system for interfacing with a plurality of objects in a three-dimensional virtual medical imaging workspace environment, the system comprising:
a processor; and
a memory with computer code instructions stored thereon, the processor and the memory, with the computer code instructions being configured to cause the system to:
identify a user intended contextual selection of a given object of the plurality of objects arranged in the three-dimensional virtual medical imaging workspace environment, based on detected gaze tracking information of the user in the three dimensional medical imaging workspace environment;
determine a command context based on the identified selected object, the determined command context comprising voice-activated commands, gesture-activated commands, or a combination thereof; and
activate an object-specific action based on a command identified from the determined command context.

14. The system of claim 13, wherein at least a subset of the plurality of objects comprises imaging study panes.

15. The system of claim 14, wherein activating an object-specific action comprises invoking a hanging protocol for a selected imaging study.

16. The system of claim 15, wherein activating an object-specific action comprises invoking an image manipulation tool.

17. The system of claim 15, wherein activating an object-specific action comprises invoking a dictation annotation tool.

18. The system of claim 15, wherein the processor is further configured to:
link a location within a selected imaging study pane to data associated with a complementary study pane displayed in the virtual environment; and
provide for display of the linked data in the complementary study pane.

19. The system of claim 13, wherein at least a subsect of the plurality of objects comprises imaging workflow panes.

20. The system of claim 19, wherein the imaging workflow panes comprise a study navigation pane, a communication pane, a reference pane, a patient data pane, or any combination thereof.

21. The system of claim 13, wherein activating the object-specific action comprises locking the identified object for primacy within the three-dimensional virtual environment based on a detected voice command.

22. The system of claim 13, wherein activating the object-specific action comprises invoking a dictation mode based on a detected voice command and terminating the dictation mode based on a detected gesture command.

23. The system of claim 13, wherein the processor is further configured to:
display to a user, the three-dimensional virtual medical imaging workspace including the plurality of objects.

24. The system of claim 13, wherein the processor is further configured to:
identify a command from the determined command context based on the detected user voice or gesture data.
